(19) United States
(12) Patent Application Publication
(10) Pub. No.: US 2002/0051769 A1
(43) Pub. Date: May 2, 2002

(54) Gene delivery vectors and their uses

(76) Inventor: Xiaoliu Zhang, Cambridge (GB)

Correspondence Address: KLARQUIST SPARKMAN CAMPBELL, LEIGH & WHINSTON, LLP, One World Trade Center, Suite 1600, 121 S.W. Salmon Street, Portland, OR, 97204, US

(21) Appl. No.: 09/839,698

(22) Filed: Apr. 20, 2001

Related U.S. Application Data
(63) Continuation of application No. 09/173,947, filed on Oct. 16, 1998, now abandoned.

(30) Foreign Application Priority Data
Oct. 17, 1997 (GB) 9721909

(51) Int. Cl. A61K 48/00; C12N 7/01; C12N 15/861

(52) U.S. Cl. 424/93.21; 435/457; 435/235.1

(57) ABSTRACT

Gene Delivery Vectors and Their Uses

Preparations of infectious viral particles include viral particles which can act as helper virus for adeno-associated virus (AAV), and include particles comprising DNA (i) that includes at least one chosen nucleic acid sequence for delivery to target host cells, and further encoding proteins and replicating functions which together are sufficient, when said particles of said preparation infect first target host cell, for assembly and release, from said first target cells of infectious recombinant AAV particles that comprise said chosen nucleic acid sequence, whereby said infectious recombinant AAV particles are able in turn to infect second target host cells, and cause expression of said DNA (i) in said infected second target host cells.

### FIELD OF THE INVENTION

This invention relates to gene delivery vectors, to processes and intermediates for their preparation, and processes in which they are used.

The invention in certain embodiments provides new preparations for the delivery of chosen DNA to target cells.

In certain embodiments, the invention also provides new high yielding processes for the production of recombinant adeno-associated virus (AAV) particles carrying desired DNA, especially heterologous DNA.

The invention also provides new preparations of recombinant AAV particles carrying chosen DNA for delivery to cells.

### BACKGROUND OF THE INVENTION AND PRIOR ART

Herpesviral amplicons are known, as well as vectors based on them; examples as well as citations to earlier published documents are given in specification WO 96/29421 (Lynxvale Ltd and Cantab Pharmaceuticals Research Ltd; S Efstathiou, S C Inglis and X Zhang). HSV amplicons retain the HSV replication origin and packaging signal orisS-pac. It is well known that HSV amplicon plasmids, once introduced into cells together with HSV as helper virus, can be amplified and packaged into HSV particles.

Adeno-associated virus (AAV) is known as a non-pathogenic human parvovirus and has been proposed for use as a gene transfer vector. Recombinant AAVs are also known, as described e.g. in U.S. Pat. No. 4,797,368 (DHHS: B J Carter & J D Tratschin): U.S. Pat. No. 5,139,941 (Univ Florida Res Foundation, N Muzyczka et al); U.S. Pat. No. 5,474,935 (DHSS: S Chatterjee and K K Wong); WO 95/06743 (UAB Research Foundation: J Dong and RA Frizzell); and U.S. Pat. No. 5,589,377 (Rhone Poulenc Rorer: J Lebkowski et al). Problems however still face development of rAAV (recombinant AAV) for gene delivery; these problems include low efficiency of current packaging systems for rAAV stock generation, and problems of purification from helper virus.

M Feng et al, Nature Biotechnology (1997) 15:866-870, describe a pair of adenoviral vectors for gene delivery and expression, so that cells infected with both vectors released recombinant retrovirus that then infected surrounding cells.

There remains a need for further gene delivery systems, especially those based on adeno-associated virus; and the present invention seeks to provide such systems, having in various embodiments features and advantages as mentioned below.

### SUMMARY AND DESCRIPTION OF THE INVENTION

According to the invention there is provided, first, an infection preparation of viral particles of a first virus type, able to act as a helper virus for production of adeno-associated virus (AAV) particles (for example herpes simplex virus or adenovirus), in which the nucleic acid component includes a chosen nucleic acid sequence for delivery to target cells, and which further encodes proteins and replicating functions which together are sufficient, in first infected cells, being cells infected by said viral preparation, to allow assembly and release of further infectious particles of a viral type, such as recombinant AAV, different from said first viral type, the further particles comprising protein and said chosen nucleic acid sequence, and able in turn to infect second infected cells and cause expression of said chosen nucleic acid therein.

In one aspect the invention provides a preparation of infectious viral particles including particles which can act as helper virus for adeno-associated virus (AAV), and including particles comprising DNA (i) that includes at least one chosen nucleic acid sequence for delivery to target host cells, and further encoding proteins and replicating functions which together are sufficient, when said particles of said preparation infect first target host cells, for assembly and release, from said first target cells, of infectious recombinant AAV particles that comprise said chosen nucleic acid sequence, whereby said infectious recombinant AAV particles are able in turn to infect second target host cells, and cause expression of said DNA (i) in said infected second target host cells.

The viral particles can consist of or include herpesvirus particles which can be replication-defective herpesvirus e.g. genetically disabled herpesvirus lacking the function of a gene essential for production of infectious new herpesvirus particles when said herpesvirus infects a normal host cell, see e.g. WO 92/05263 (immunology Ltd; S C Inglis et al) and WO 94/21807 (Cantab Pharmaceuticals; Inglis et al). A normal host cell is generally a cell that does not contain recombinant elements intended to supplement defective virus functions, which are non-native to cells of the host cell's parental type. Such replication-defective recombinant AAV particles as described and referred to herein, containing heterologous DNA for delivery to a target cell, are normally not capable of giving rise to a further generation of AAV particles when they infect a target cell, which is usually a normal host cell, e.g. a cell of a tissue of a treated human or non-human mammal.

The invention in certain embodiments can provide or contribute towards the following aims.

Examples of the present invention can provide targetting of chosen DNA to a range of host target cells that surround the host target cells initially infected by the vector preparation, so as to allow expression of said DNA in said surrounding cells.

Examples of the system can provide gene delivery and expression of chosen DNA in cells which do not initially become infected by viral particles of the preparation itself.

Examples of the system can enable expression of chosen DNA in cells that do not themselves become infected by viral particles of the administered preparation itself, and therefore are not infected by viral particles of the same type as the particles of the administered preparation, and therefore do not become the target, or not the primary target, of any immune response against the viral particles of the administered preparation. e.g. an anti-herpes immune response.

Examples of the invention enable rAAV infection to take place in the absence of systemic administration of rAAV particles, and this can help in initial escape at least of the primary target cells from an anti-AAV immune response.

Examples of the invention can provide rAAV products free or virtually free of helper virus particles, e.g. free of replication competent helper virus particles.

Examples of the technique are applicable to single-particle delivery of more than one component that is to be delivered to target cells.

Examples of the technique are applicable to providing immunostimulation by gene delivery of cytokine genes without the need to transfect/infect longlived cells with cytokine genes, i.e. by ensuring that the cells that express the immunostimulant are cells infected by a virus infection, e.g. with rAAV, that will ensure cell death.

Examples of the technique are applicable to monitoring of expression of gene(s) delivered by the vector preparation, e.g. when the heterologous DNA of the vector preparation comprises a reporter gene.

Preparations according to examples of the invention are applicable to gene delivery over a usefully wide host range.

Examples of the preparations can give a useful yield of rAAV or its second-generation expression product in about 24-48 hours from infection by herpesvirus or amplicon.

Alternative examples of the preparations can be based on recombinant adenovirus.

Examples of preparations according to the invention can be used in therapy, to infect cells in-vivo and produce expression of such chosen nucleic acid, e.g. to express an antigen for which an encoding gene is present in the preparation, in order to evoke an immune response. Gene therapy techniques provided by examples of the invention can for example be corrective gene therapy or gene delivery techniques to replace a defective or missing gene in a target cell, or can be gene immunotherapy techniques to express a gene intended to evoke or modulate a desired immune response.

Chosen DNA for delivery to and expression in target cells can comprise DNA encoding one or more heterologous genes, e.g. genes encoding antigens, such as tumour specific antigens, or encoding cytokines or other immunostimulatory or other immunomodulatory proteins; e.g. as mentioned and cited in WO 96/26267 (Cantab Pharmaceuticals; Inglis et al). The chosen DNA can if desired encode a therapeutic gene, e.g. a gene intended to be delivered and expressed to correct a genetic deficiency in a target cell or tissue. The chosen DNA can also optionally encode a regulatory DNA sequence, such as a transcription factor, or a tissue specific promoter sequence (for example the albumin promoter which directs liver specific gene expression, or the neuron enolase promoter which directs neuron specific gene expression), providing the size of the complete heterologous DNA insert is of a size capable of being packaged into rAAV particles, e.g. usually up to about 1.5 kb. The presence of a tissue specific promoter sequence to direct expression of target DNA to specific cell types can be highly useful, as adeno-associated virus vector itself has a wide tissue tropism.

In certain embodiments, the invention provides for example a preparation of viral particles of a first virus type which is a herpesviral preparation or a herpesviral amplicon preparation which

(a) is replication-defective as regards production of infectious new herpesviral particles or new herpesviral amplicon particles, and which

(b) comprises (i) DNA which is heterologous to AAV, e.g. up to about 4.5 kb in size, and flanked by ITR sequences of AAV, and (ii) DNA encoding AAV rep and cap genes coded at least in part in a position other than flanked by AAV ITR sequences.

wherein both (i) and (ii) are positioned in relation to a herpesviral origin of replication (oriS) and optionally also a herpesviral packaging signal (pac) so that they are replicatable within a cell infected by the preparation,

such that when first cells are infected with said preparation they cannot give rise to infectious new particles of herpesvirus or of herpesviral amplicon, but they can give rise to recombinant AAV particles comprising DNA as described above at (i), packaged in AAV coat protein, constituting recombinant AAV particles,

and such that said recombinant AAV particles, after their release from said first cells, can infect second cells, but can not give rise to infectious new virus particles from said second cells.

In such a preparation, the DNA (i) and the DNA (ii) can be encoded by the same or different herpesviral amplicons including oriS and pac sequences. The result in such examples, can be that the gene(s) for delivery can all be encoded in amplicon DNA.

The DNA (i) and the DNA (ii) can for example be encoded in the same herpesviral amplicon.

Alternatively, DNA (i) can be encoded by a first herpesviral amplicon and DNA (ii) encoded by a second herpesviral amplicon.

It is not necessary to use amplicons, however. An alternative kind of preparation according to the invention can comprise DNA (i) indicated above and the DNA (ii) indicated above, both encoded by an infectious mutant herpesvirus (e.g. respective different mutant herpesviruses for DNA (i) and (ii)), that has a mutant genome lacking a gene essential for production of infectious new herpesvirus particles, but generally not essential for expression of herpesviral proteins in a cell infected by the mutant herpesvirus.

In a further alternative, rep and cap can be encoded in a DISC herpesviral mutant, i.e. one that lacks a gene essential for production of infectious new herpesvirus particles, mutant herpesvirus, with other DNA for delivery encoded in an amplicon; thus, DNA (i) can be encoded by a herpesviral amplicon and DNA (ii) encoded by a DISC herpesviral mutant, or vice versa.

The elements of (i) and/or (ii), where they are encoded in the DISC herpesvirus mutant, can be inserted at the site of deletion of the essential herpesviral gene.

In certain important examples, an immunostimulatory gene e.g. a gene encoding IL-2 or another cytokine, or other immunomodulatory gene, or a gene encoding an antigen, or a gene encoding a therapeutic protein, e.g. encoding Factor VIII or IX, for delivery to and expression in a cell lacking such gene, and/or a reporter gene, e.g. gfp or Lac Z, can also be inserted, especially for example in the mutant herpesvirus or herpesviral amplicon(s).

Certain examples of the invention can incorporate integration functionality, e.g. in the form of a preparation as described above, where DNA (i) which is heterologous to AAV, e.g. up to about 4.5 kb in size, and flanked by ITR sequences of AAV, is accompanied by the AAV rep gene or by a sub-sequence of the rep gene that is sufficient to cause integration of said DNA (i) into the DNA of said second cell infected by the recombinant AAV particles.

According to an aspect of the invention, there is also provided a preparation of recombinant AAV (adeno-associated virus) genomes comprising heterologous DNA and packaged in AAV coat protein, which can for example be free of helper virus, and/or free of adenovirus. The preparations can for example be free of infectious replication-competent helper-virus. They can contain replication-defective herpesvirus but are preferably free of replication-competent herpesvirus.

Also provided by the invention is a method of producing recombinant AAV genomes, e.g. one that is free of replication-competent helper virus, comprising heterologous DNA and packaged in AAV coat protein, comprising the steps of:

(i) providing a herpes virus with a genome that lacks a gene that is essential for production of infectious new herpesvirus particles, but not essential for the general expression of herpesviral proteins, and which has been grown by culture on cells made recombinant and able to express the function of the viral gene that is lacking in the herpesviral genome;

(ii) providing a herpesviral amplicon that comprises a rep and cap gene of AAV, and ITRs positioned so as to flank heterologous DNA that is desired to be incorporated in a recombinant AAV particle;

(iii) using the herpesvirus from (i) and the amplicon from (ii) to infect cells that do not express the function of the gene that is lacking in the herpesviral genome, and

(iv) harvesting from the cells infected in (iii) said recombinant AAV genomes comprising said heterologous DNA and packaged in AAV coat protein, preferably free of replication-competent helper virus.

The invention also provides various preparations of recombinant AAV particles, for example: a preparation of recombinant AAV genomes comprising heterologous DNA e.g. up to about 4.5 kb in size, flanked by ITR sequences of AAV and packaged in AAV coat protein, e.g. free of helper virus, and/or free of adenovirus, and/or free of infectious replication-competent helper virus, and/or containing replication-defective herpesvirus but free of replication-competent herpes virus or other helper virus,

The invention also provides processes for producing recombinant AAV particles, for example a method of producing recombinant AAV genomes (e.g. free of replication-competent helper virus) comprising heterologous DNA e.g. up to about 4.5 kb in size, flanked by ITR sequences of AAV and packaged in AAV coat protein, in which the process comprises the steps of

(i) providing a herpes virus with a genome that lacks a gene that is essential for production of infectious new herpesvirus particles, but not essential for the general expression of herpesviral proteins, and which has been grown by culture on cells made recombinant and able to express the function of the viral gene that is lacking in the herpesviral genome;

(ii) providing at least one herpesviral amplicon comprising in addition to a rep and cap gene of AAV, ITR sequences of AAV positioned so as to flank heterologous DNA that is desired to be incorporated in a recombinant AAV particle;

(iii) using the herpesvirus from (i) and the amplicon from (ii) to infect cells that do not express the function of the gene that is lacking in the herpesviral genome, and

(iv) harvesting from the cells infected in (iii) said recombinant AAV genomes comprising said heterologous DNA end packaged in AAV coat protein, preferably free of replication-competent helper virus.

According to alternative embodiments of the invention, examples such as those with features as described above can be carried out using adenovirus as helper virus, instead of herpes virus.

Examples of processes and materials useful in connection with carrying out the present invention are further described below, by way of illustration but not for limitation.

Examples of cell lines and viruses usable in connection with the invention are as follows:- Vero and BHK cells are obtainable from the European Collection of Animal Cell Cultures (ECACC no.88020401 and no.85011423 respectively; Porton Down, UK). Construction of CR-1 and BHK.TK.gH⁺ cells has been described by M F G Boursnell et al, (1997) J Infect Dis 175(1) pp16-25; and X Zhang et al, (1995) J gen Virol 79(1) pp125-131. These cell lines incorporate the gene encoding gH from HSV-1 and can therefore serve as complementing cells for growing gH-deleted HSV which, in non-complementing cells, is a disabled infectious single-cycled virus (DISC-HSV). CR-1 cells can be grown in DMEM foetal calf serum (FCS) and BHK cells can be grown in Glasgow Modified Eagle's Medium supplemented with 5% tryptose broth (GMEM) and 10% FCS. 293 cells are obtainable from ATCC (CRK 1573) and can be grown in DMEM supplemented with 10% FCS. HSV-1 strain SC16 is a well-known clinical isolate. Among the genetically disabled HSV strains that can be used as helper virus for generating both rAAV and amplicon stocks, is a deletion mutant derived using per-se known technique from HSV-1 strain SC16, and having a deletion that covers the gH region and part of thymidine kinase (TK) gene. DISC virus stocks can be grown and titrated on CR-1 cells. A mutant Ela E3-deleted adenovirus is a wellknown mutant, and can be grown and titrated on 293 cells.

Gene Delivery In Vitro:

Gene delivery using vector constructs according to embodiments of the invention can be carried out for example as follows:

Gene delivery using the vectors described herein can for example be carried out in vitro, for example by using DISC-AAV-gfp, a recombinant disabled herpes virus containing rAAV sequences and also the gfp reporter gene; or by usng pHAV6.6 amplicon, an AAV amplicon plasmid carrying rep and cap genes and ITRs, and which also contains the GFP gene. Construction of both of these vectors is described in more detail herein. The GFP gene is useful for investigative purposes, and for other purposes analogues of the vectors can readily be constructed using corresponding desired genes other than GFP, e.g. a therapeutic gene, e.g. a gene encoding factor IX (fIX).

Vero cells can be infected with 5 pfu/cell of DISC-AAV-gfp or 5 infection unit (IU) per cell of pHAV 6.6 amplicon stock. The infection step can be carried out for 20 minutes at 37° C. The cells can then be spun down and washed twice with 50 ml of medium. The infected cells can be resuspended and mixed with uninfected Vero cells at a ratio of 1:100. The mixed cells can be re-seeded back into tissue culture containers with medium containing 0.5% FCS. The cells can be checked with UV fluorescent microscopy, e.g. daily, for the appearance of green fluorescence and the spreading of fluorescence to neighbouring cells. At day one after the mixed cells are re-seeded, many single fluorescent cells can be seen scattered across the mostly dark field of the fluorescence microscope. These cells are normally those originally infected by the GFP containing HSV vectors, and can be termed seed cells. These cells can go on to produce rAAV progeny. At day two, the fluorescent pattern is similar to that seen on day one. However, at day three after the cell seeding, the cells surrounding those seed cells can be seen to start to show green fluorescent emission. This fluorescence diffusion occurs at the cells across multilayers of the neighbouring cells, with the cells closer to the seed cells have the strongest fluorescent emission. Under the microscope the overall fluorescent pattern of a number of test specimens has appeared to have somewhat the shape of a bunch of grapes. This fluorescent pattern may be seen to remain unchanged for the next few days if a test culture is maintained without passaging, and before the life of the cells in the unpassaged test culture expires.

Both the recombinant DISC-AAV-gfp and the amplicon pIIAV-6.6 are without the property of cell-to-cell spreading in Vero cell culture: the green fluorescence spreading seen in the test arrangement just described above is evidence of the spreading of rAAV produced from the cells designated above as seed cells, rAAV which then enters into the neighbouring cells and delivers the GFP gene into these cells.

Further confirmation of this can be gained by tests in which vectors containing rAAV, but without a rep-cap sequence, can be employed to carry out the corresponding experiment. In this case no rAAV is produced from those cells initially infected by analogues of either the DISC-AAV-gfp or pHAV-6.6 which lack rep and cap genes. In tests carried out up to the present application, spreading of fluorescence as in the experiment described above is not seen. As expected, no fluorescent diffusion is observed for up to 5 days after the initial mixed cell culture is set up. This result further confirms the conclusion that rAAV produced from the first-stage infected seed cells (using the rep I cap+vectors) spreads to the surrounding cells. Thus two-stage gene delivery can be shown in vitro.

Two Stage Gene Delivery In Vivo:

Two-stage gene delivery can be shown in living tissue by carrying out an analogue of the preceding procedure in vivo using the mouse as animal model.

An ex vivo procedure can be carried out as follows: In this procedure, a mouse fibroblast cell line L920 can be infected in vitro with 5 pfu/cell of either DISC-AAV-gtp, DISC-AAV-fIX, or amplicon constructs pHAV-6.6 and pHAV-6.8, respectively. The infection can be done at 37° C. for 20 minutes. Then the cells can be thoroughly washed with medium. Afterwards the treated cells can be injected into mice subcutaneously. Gene expression can be monitored either for example by examining tissue sections for GFP under the UV fluorescent microscopy in the case of treatment with constructs containing GFP gene, or through immunohistochemistry staining for factor IX protein in the case of treatment with constructs containing factor IX gene. The factor IX gene expression can also be monitored by routine ELISA assay for factor IX on blood samples collected from experimental animals. In vivo two-stage gene delivery can be shown either by the appearance if a similar GFP diffusion pattern as that shown in vitro, or by long-term factor IX expression in the blood collected from the animals.

Utilisation of DISC AAV and Amplicon-AAV for rAAV Stock Generation:

DISC-AAV and amplicon-AAV constructs as disclosed herein can be used for generating high titre recombinant adeno-associated virus (rAAV) stocks which can be free or virtually free of helper virus contamination.

A problem facing development of rAAV (recombinant AAV) for human gene therapy has been low efficiency of current available packaging systems for rAAV stock generation. A currently used method for generating rAAV vectors comprises co-transfection of a recombinant vector containing rAAV sequence together with a packaging plasmid, into cells such as well-known and available 293 cells. The cells are subsequently infected with adenovirus, to serve as a helper virus for AAV lytic phase of infection. The vector plasmid can contain a gene of interest and the transcription control elements, flanked by ITRs. The packaging plasmid can contain entire AAV genome sequences except the ITR sequences. In transfected cells the rAAV genome flanked by the ITRs is excised, replicated, and encapsidated into viral particles composed of cap proteins provided in trans from the packaging plasmid. The helper virus used in this packaging system has been for example adenovirus E1 deletion mutant.

Problems with such a current packaging system include: (1) It can be difficult to generate stocks with high titre of rAAV. A typical yield of rAAV from this system can be approximately 10 colony-forming units (cfu) per 10-cm culture plate. Therefore, to obtain a sufficient amount of rAAV for a routine study, typically more than 100 plates may need to be transfected which can be labour-intensive and time-consuming. The harvested virus may have to be concentrated by column chromatography so that the titre may be high enough for use. (2) The stocks generated in this way cannot normally be expanded by further passage. Each stock normally has to be generated ab Initio, i.e. by transfection. (1) Contamination of infectious helper adenovirus (with potential contamination of wt adenovirus): can mean in practice that extensive separation procedures are required following initial stock preparation. Routinely, three rounds of buoyant density ultracentrifugation can be required to remove contaminated helper virus to an undetectable level. Such a purification procedure not only is time-consuming, it can also cause significant loss of rAAV titre.

A difficulty appearing to stand in the way of establishing packaging cell lines for rAAV appears to lie in cytotoxicity of rep proteins. Recently, cell lines expressing rep under inducible promoters (methallothionein promoter or Ad inducible promoter) have been reported. Even in such a cell line, helper adenovirus is still required and therefore the contamination of helper virus remains a problem.

According to certain embodiments of the present invention, genetically disabled herpesvirus such as virus as described in WO 92/05203 (Immunology Limited: Inglie et al) can be used as helper virus for rAAV (instead of adenovirus, as both adenovirus and HSV can serve as helper for AAV lytic infection) and herpesviral amplicons or disabled herpesvirus can carry genes encoding AAV rep and capsids to provide (In trans) the function of rAAV packaging. Initial rAAV stocks can be generated through transfection or viral infection, e.g. and preferably by transfection with amplicon plasmid DNA in complementing cells for DISC-HSV, such as BHK or Vero complementing cell lines. Such a stock, according to an aspect of the invention, can readily be expanded by subsequent passages through infecting more complementing cells, and this can often provide advantage in production. Such stocks can then be eventually passaged in non-complementing cells for the defective herpesviral helper, such as (non-complementing) Hela or Vero cells. After a single passage in such non-complementing cells, the packaging of rAAV is not affected, but the infectious defective herpesvirus and amplicons can in such a stage readily be got rid of, so that a stock can be prepared which is free or virtually free of infectious helper virus particles. It is envisaged that this system can overcome problems such as those mentioned above,

An example of a detailed experimental procedure useful for making embodiments of the present invention is as follows:

rAAV stocks can be generated from amplicon constructs as follows: BHK,TK-, GH+ cells can be transfected with pHAV-5.8 by lipofectamine (from Gibco BRL), 2 μg of amplicon plasmid DNA can be added to 200 μl sterile H₂O and 10 μl of lipofectamine can be diluted by 20 fold with sterile H₂O. The thus-obtained DNA and lipofectamine solutions are mixed together gently and left at room temperature for 30 minutes. Then 1.6 mls of Optimem medium (as supplied by the manufacturer) can be added to the DNA-lipofectamine mixture. Cells can be rinsed with serum-free medium and the DNA-lipofectamine mixture can be overlaid gently on to the cells. After incubation for 5 hours at 37° C., the transfection solution can be removed and replaced with 5 mls of GMEM plus 5% FCS. Cells can then be incubated at 37° C. for another 16 hours. The resulting cells can then be infected with 1 pfu/cell of genetically-detective HSV, (particularly (where gH+ cells are used) gH HSV1 virus such as that described herein or in WO 92/05263), for another 24 hours. Viruses can be harvested and titrated by plaque assay for the titre of the helper virus. This virus stock can be further passaged in BHK, TK, gH⁺ cells 2 3 times with the last passage In Vero cells. For each passage, cells can be infected with 1 pfu/cell of virus (based on the titre of PS1). Then the rAAV titre can be titrated e.g. for assay purposes by infecting Vero cells with a serial diluted virus solution and the GFP positive cells can be counted under UV fluorescent microscopy. Preliminary results have shown that rAAV with a titre as high as 1×10⁹ can be generated from the first step, i.e. transfection of pHAV-6.6 followed by super-infection of gH- HSV1,

rAAV stocks can also be generated from gH- HSV -AAV recombinant virus, e.g. as follows: Vero or Hela cells can be infected with gH- HSV1 containing AAV-gfp sequences (described below) at 1 pfu/cell for one hour. Then medium containing 1% FCS is added and the infection is left for another 24 hours. Virus harvest is titrated by plaque assay for the appearence of DISC-HSV. The rAAV titre can be quantified in a similar way as described above.

Vector construction for suitable recombinant herpesviruses can be carried out as follows:

Recombinant adeno-associated virus AAV can be obtained using as starting material plasmid pAV1, which contains the entire AAV-2 genome, and is publicly available on a commercial basis from The American Type Culture Collection, Rockville, Md., under deposit number ATCC No. 37215. The following rDNA manipulations can be performed in per-se known manner.

The entire AAV-2 genome can be excised out of plasmid pAV1 with restriction enzymes BgIII and PvuII and cloned into plasmid puc119. The resulting plasmid is designated pucAV1. Plasmid pucAV1 can be digested jointly with SnaBI and PpuMI, to remove the AAV coding sequence but to leave both the 5′ ITR (nucleotides 1-191 of AAV sequence) and the 3′ ITR (nucleotides 4494-4675) intact.

The resulting sequence can be used to construct a plasmid containing a rAAV sequence with a marker gene encoding green fluorescent protein (GFP). Plasmid pEGFP-N1, containing an enhanced version of GFP, is commercially available from Clontech (cat: 6085-1). A 3766 bp DNA fragment can be generated from joint digestion of pEGFP-N1 using AseI and BsaI, and contains CMV promoter-GFP-polyA as well as the neomycin cassette. This can be cloned by blunt-end ligation into pucAV1 which has been digested as described above with SnaBI and PpuMI, so that the GFP and neomycin containing DNA fragment is flanked by AAV ITRs. This resulting plasmid is designated pTR-CMVgfp.

The digestion product of pucAV1 can also be used to construct a plasmid containing rAAV together with a therapeutic gene of choice. A therapeutic gene such as the (per-se known and available) gene encoding clotting factor IX (for treating haemophilia B) can be cloned into the rAAV ITR cassette in an analogous way as described above for GFP. Where this is carried out using the gene for factor IX, the resulting plasmid can be designated pTR-fIX.

More generally, rAAV sequences can be made as DNA fragments which contain, in the following order, AAV 5′ LTR, a suitable mammalian promoter of choice, a gene of interest (such as the gene for GFP or for factor IX in the examples just described), a poly-A signal and a AAV 3′ LTR.

HSV amplicon plasmids containing rAAV sequences can be constructed as follows:

rAAV sequences, made as described above, with (for example) either GFP or factor IX, can be excised out of the respective plasmids carrying them by digestion with both pVUI and AseI, and blunt-end ligated into the unique SapI site in the amplicon plasmid pW7TK which can be obtained as described in patent application WO 96/29421 (Lynxvale Ltd and Cantab Pharmaceuticals Research Ltd: Efstathiou, Inglis and Zhang). The resulting amplicon plasmids can be designated pHAV-5.6 and pHAV-5.8, respectively.

HSV amplicons containing both rAAV sequences and AAV rep and cap coding sequences can be constructed for example as follows:

Suitable plasmids can be constructed as follows: The AAV sequences covering the region for coding rep and cap gene products can be cut out from pAV1 by Ball digestion and cloned into the ScaI site of pHAV-5.6 and the unique SapI site of pHAV-5.8. The resulting amplicon plasmids can be designated pHAV-6.6 and pHAV-6.8, respectively.

Amplicon stocks can be generated as follows: Initially amplicon plasmid DNA from either pHAV-6.6 or pHAV-6.8 can be transfected into CR 1 cells (gH+ recombinant complementing mammalian cells capable of hosting gH- HSV1 virus) either by calcium phosphate precipitation or by lipofectamine (from Gibco BRL). Cells can be seeded one day before transfection in 5 cm petri dishes. For calcium phosphate precipitation, 8 μg of DNA can be mixed with 0.5 ml Hepes buffered saline (HEBS) pH 7.05 and 70 μ1 of 2M CaCl₂ at room temperature. for 30 minutes. The culture medium can be removed and the DNA precipitate added to the cells. The cells can be incubated at 37° C. for 40 minutes before removal of the transfection mixture and its replacement with 4 mls of GMEM plus 5% FCS. Cells can be incubated at 37° C. for another four hours before being treated with 1 ml of 25% DMSO in HEBS for exactly 4 minutes. The DMSO solution could then be removed and cells washed with serum-free medium. 5 ml of GMEM plus 5% FCS can be added and the cells incubated at 37° C. for another 16 hours. The lipofectamine transfection can be carried out according to the supplier's instructions: 2 μg of amplicon plasmid DNA can be added to 200 μl sterile H₂O and 10 μl of lipofectamine is diluted by 20 fold with sterile H₂O. The DNA and lipofectamine solutions can be mixed together gently and left at room temperature for 30 minutes. Then 1.6 mls of Optimem medium (as supplied by the manufacturer) can he added to the DNA-lipofectamine mixture. Cells can be rinsed with serum-free medium and the DNA-lipofectamine mixture overlaid gently onto the cells. After incubation for 5 hours at 37° C. the transfection solution can be removed and replaced with 5 mls of GMEM plus 5% FCS. Cells can be incubated at 37° C. for another 16 hours and then infected with 1 pfu/cell of PS1 for another 24 hours. Viruses can then be harvested and titrated by plaque assay. This virus stock can be further passaged in BHK, TK, gH⁺ cells 2-3 times. For each passage, cells can be infected with 3-5 pfu/cell of virus (based on the titre of PS1) for 1 hour. Then a selection medium containing 0.6 mM methotrexate and 1×TGAG (40×TGAG: 0.6 mM Thymidine, 3.8 mM Glycine, 9 mM Adenosine, 1.9 mM Guanosine) can be added and the infected cells cultured at 37° C. for 24-28 hours before harvesting and if desired titration.

Recombinant gH- HSV1 virus containing both rAAV and AAV rep-cap coding sequence can be constructed as follows:

As an intermediate stage in the construction of examples of vectors according to the present invention, it can be convenient to construct deletant HSV virus lacking an essential gene, e.g. gH- HSV-1. In th present example, a deletant HSV1 is made which lacks the HSV1 gH gene, and contains a PacI site at the gH locus, to facilitate later insertion of desired heterologous DNA into the locus of the deleted gH gene. A PacI restriction site is be preferred here for its convenience because it does not cut the wild-type HSV1 genome. Such a mutant gH- HSV1 can be used for present purposes either for carrying rAAV and rep cap sequences, or as a helper virus for amplicon stock generation. Foreign DNA to be inserted is flanked (using per-se known rDNA manipulation techniques) by restriction sites corresponding to a convenient restriction site chosen and inserted if necessary at the proposed insertion site in the virus (here it is a PacI site), and can then be ligated into the gH- HSV1 genome to generate a desired new recombinant virus. (Vaccines based on genetically defective herpesvirus lacking a gene essential for production of infectious new virus particles are described in WO 92/05263 (Immunology Limited: Inglis et al) and more recent publications. The gH gene is a preferred example of an essential glycoprotein H (gH) gene for deletion in such a mutant virus. See also Forrester et al., J. Virol. 66, 341-348. 1992. An infectious stock of gH- HSV1 can be grown in a complementing cells which express endogenous gH, also described in the cited documents. Progeny virus particles can result from infection of normal cells (i.e. non-complementing cells not made to express viral gH) but these are not infectious.)

Plasmids to be used for generation of a gH- HSV1 deletion mutant can be constructed as follows:

Initially, flanking sequences to either side of the HSV gH gene can be amplified from HSV-1 strain KOS (M) viral DNA by the polymerase chain reaction (PCR), using Vent DNA polymerase, and the products cloned into EcoRI-HindIII-cut pUC119. The resultant plasmid can be designated pIMMB25, and details of a further plasmid to be used can be as for plasmid pIMMB26 described in WO 96/29121, cited above, and incorporated herein by reference). A synthetic oligonucleotide comprising sequences JM1 Oligo 5′ CGA TTA ATI AAG TTA ACT AGA AGA CAA IAG CAG GCA TGC TGG GGA TGC GGT TAA TTA AGA3′; and JM2 Oligo 5′ TCT TAA TTA ACC GCA ICC CCA GCA TGC CTG CTA TTG TCT TCT AGT TAA CTT AAT TAA TCG 3′), which contains in its middle two PacI restriction enzyme sites, can be cloned into the HpaI site of pIMMB25, which in turn is located in the middle of the gH flanking sequences. The resulting plasmid can be designated pIMMB-IacZPac. A DNA cassette containing CMV promoter, IacZ gene and a polyA signal can be cut out from pAMP-IacZ-ci and cloned into the HapI site in pIMMB-LacZPac so that it is flanked by the PacI sites. The resulting plasmid can be designated pIMX-18.

Recombinant virus can be constructed by transfection of type I HSV (strain sc16) viral DNA with plasmid pIMX-18. Viral DNA can be purified on a sodium iodide gradient (as described in (1976) Virology 74, 256-258). Recombination can be carried out as follows: a transfection mixture can be prepared by mixing 5 μg of viral DNA. 0.5 μg of linearised plasmid DNA (linearised by digestion with the restriction enzyme Scal) in 1 ml of HEBS buffer (137 mM NaCl, 5 mM KCl, 0.7 mM Na₂HPO₄, 5.5 mM glucose. 20 mM Hepes, pH 7.05). 70 μl of 2M CaCl₂ can be added dropwise, and mixed gently. The medium can be removed from a sub-confluent 5 cm dish of CR1 or CR1 cells and 500 μl of the transfection mixture added to each of two dishes. The cells can be incubated at 37° C. for 40 minutes, then 4 ml of growth medium containing 5% foetal calf serum (FCS) added. 4 hours after adding the transfection mix, the medium can be removed and the cells washed with serum-free medium. The cells can then be ‘shocked’ with 500 μl per dish of 15% glycerol for 2 minutes. The glycerol is then removed, the cells washed twice with serum-free medium and growth medium containing 5% FCS added.

After 4-7 days or when a full viral cytopathic effect (CPE) is observed, the cells can be scraped into the medium, spun down at 2500 rpm for 5 minutes at 4° C., and resuspended in 120 μl of Eagles minimal essential medium (EMEM). This now yields a crude virus stock containing wild-type and recombinant virus.

The stock can be frozen, thawed and sonicated and screened for recombinants on CR1 cells at a usual range of dilutions. After addition of the virus dilutions, the cells can be overlaid with medium containing 1% low-gelling-temperature agarose. After the appearance of viral plaques at about 3 days, a second overlay of agarose containing 330 μg/ml of Xgal can be added. Blue plaques can be picked, within 48 hours, and transferred to 24-well dishes (1 cm² per well) containing CR1 cells. The plaques can be allowed to grow to full CPE and harvested by scraping into the medium. Multiple rounds of plaque-purification can be carried out until a pure stock of virus is obtained or other desired stage of purification reached.

The structure of the recombinant can be confirmed as follows: Sodium iodide purified viral DNA can be prepared as before, and digested with BamHI. This digest can be separated on an agarose gel and transferred to a nylon membrane. This is probed with a radiolabelled DNA fragment homologous to the sequences either side of the gH gene.

The gH- HSV1 virus so obtained, containing a single PacI site at its gH locus, can be designated DISC-HSVPac. Insertion of both rep-cap and rAAV into DISC-HSVPac virus can be carried out as follows. A plasmid containing double PacI sites can be made with the help of the same linker as used to construct DISC-HSVPac, which is first ligated into pRC/CMV cut with NruI and SacI. The resulting plasmid can be designated pIMJ1.

A rep-cap sequence can be cloned into pIMJ1 as follows: The BaI fragment of a plasmid such as pAV1 (which contains the entire rep-cap sequence of AAV-2) can be blunt-ended by polymerase treatment and ligated into the unique HpaI site of pIMJ1 so that the rep-cap sequence is flanked by PacI sites in the new plasmid. This plasmid can be designated pIMJ-repcap.

A plasmid containing both rAAV and rep-cap sequences flanked by PacI sites can then be made by cutting out rAAV sequences containing either GFP or factor IX from plasmids pTR-CMVgfp and pTR-fIX, using restriction enzymes PvuI and AseI, and blunt-end ligated into the BbsI site in pIMJ-rep-cap so that the rAAV cassette is next to the rep-cap sequence and is also flanked by the PacI sites. These plasmids are desingated pAAV-Pac-gfp and pAAV-Pac-fIX, respectively.

rAAV and rep-cap sequences can be inserted into DISC-HSVPac virus as follows: The rAAV-repcap cassette can be cut out from pAAV-Pac-gfp or pAAV-Pac-fIX and ligated into DISC-HSVPac virus. The entire procedure can be analogous to the insertion method described above. Thus, for example, DISC-viruses containing rAAV sequences can be made by inserting rAAV vector sequences into the gH locus of DISC HSV. For example a GFP cassette flanked by AAV TRs can be removed from pTR-EGFP (as described above) by digestion with PvuI and BsaI, and cloned into a plasmid pIMJ-1 so that the AAV vector sequence becomes flanked by the recognition sequences for the restriction enzyme PacI. The entire AAV vector sequence can then be excised with PacI, and cloned e.g. into a DISCPac virus at the gH (deletion) locus. The ligated DNA can be transfected into CR-1 cells, and recombinant virus can be recovered by sequential plaque purification. Viral progeny can be screened for recombinant virus by Southern hybridisation with probes made from both rep-cap DNA and the DNA sequence containing GFP, or factor IX, as the case may be. The newly constructed recombinant viruses are designated DISC-AAV-gfp (containing GFP derived from a respective rAAV cassette) and DISC-AAV-fIX (containing factor IX gene derived from a respective rAAV cassette).

Virus stocks can be stored at −70° C., and can be used in the preparations and methods mentioned above.

Recombinant AAV via Adenovirus:

In alternative examples of the invention, vector construction for suitable recombinant adenoviruses for use as helper viruses and vectors for generating rAAV can be carried out as follows:

rDNA manipulations can be performed in per-se known manner;

Adenovirus type 2 can be obtained from the ATCC (Cat no.VR-846) and used to infect for example HeLa cells, and the DNA isolated from infected cells by phenol extraction followed by ethanol precipitation. The Ad-2 DNA can then be digested with restriction enzyme SacII, and a resulting 358 bp fragment isolated using gel electrophoresis. This 358 bp fragment, containing the Ad-2 replication origin and packaging signal, can then be ligated into the unique SmaI restriction site of plasmid pneb-193 and the resulting plasmid designated pADV-1, Plasmid pncb-193 is available from New England BioLab under catalogue number 305-1.

Recombinant AAV sequences and AAV rep and cap genes can then be inserted into plasmid pADV-1 as follows:

Plasmids pTR-fIX (described above) and pTR-EGFP contain rAAV sequences, and these rAAV sequences can be isolated by digestion of either of these plasmids using restriction enzymes PvuI and AseI, Plasmid pTR-fIX can be constructed as described earlier and plasmid pTR-EGFP can be constructed as follows: the GFP cassette can be removed from plasmid pEGFP-N1 (Clontech, Cat. No. 6085-1) and cloned into puc AV1 using PvuI and BsaI, followed by complete digestion with PpuMI and SnaBI to create pTR-EGFP. The resulting AAV sequence can then be cloned by blunt-end ligation into the unique BamHI site of plasmid pADV-1. The resulting plasmid can be designated pADV-2.

The AAV sequences covering the region coding for rep and cap gene products can be cut out from pAVI by BalI digestion and blunt-end cloned into the unique BemHI site of pADV-1. The resulting plasmid can be designated pADV-3.

Recombinant AAV stocks can be generated as follows: Initially 293 cells can be seeded one day before transfection in 5 cm petri dishes. The 293 cells can then be transfected with plasmids pADV-2 and pADV-3 using Lipofectamine™, according to the supplier's instructions as described above. Twenty-four hrs after plasmid transfection, the 293 cells can be infected with Ad-2 helper virus at a multiplicity of infection (MOI) 2. Finally, about 2-3 days after Ad-2 helper virus infection, the 293 cells yield a crude virus stock containing rAAV and contaminating adenovirus. The stock can be subjected to heating at 56° C. for one hour to inactivate contaminating adenovirus. The resulting rAAV titre can be determined by infecting a monolayer of HeLa cells with serial dilutions of samples of rAAV stock produced, followed by adenovirus infection at a multiplicity of infection (MOI) 2.

Other constructs can also be made and used according to the invention.

Further examples of plasmid and recombinant virus constructions based on recombinant herpesvirus and herpesviral amplicons that can be made and used in connection with the preparations and methods mentioned above include:

Further constructs of rep and cap genes for use in the techniques can be made as follows: pucAV1 can be partially digested with PpuMI (at two sites, one at nt 191 and another one at nt 351) and then completely digested with SnaBI. The PpuMI-SnaBI fragments which contain rep and cap sequences can be isolated and cloned either into the SapI site of pW/TK (an amplicon plasmid) or into the HpaI site of pIMJ-1 (a non-amplicon, puc119 derived plasmid). These newly constructed plasmids can be designated pIIAV-7.3 (amplicon containing the full length 4303 bp PpuMI-SnaBI fragment, and intact rep and cap genes), pIIAV-7.3 del (amplicon containing the truncated 4143 bp fragment, N-terminally-deleted rep and intact cap) and pHAV-5.6 (non-amplicon plasmid containing intact rep and cap genes), respectively.

In a further procedure for construction of AAV vector plasmids, the GFP cassette can be removed from pEGFP-N1 (CLONTECH. Cat No:6085-1) with AseI and BsaI and cloned into pucAV1, fully digested with PpuMI and SnaBI to create the plasmid pTR-EGFP. The sequence encoding GFP and flanked by AAV TRs can be removed from pTR-EGFP with PvuI and BsaI and cloned into either pHAV-7.3 to create pHAV-4.1 (amplicon containing both AAV vector sequence and rep and cap genes) or into the SapI site of pW7-TK to create pHAV-5 (amplicon containing AAV vector sequence only). To construct a non-amplicon plasmid containing both AAV vector sequence and rep-cap genes, the full length 4303 bp PpuMI-SnaBI can be cloned outside the TR sequences of pTR-EGFP to create pHAV-9.8.

Production of rAAV

For small scale rAAV preparations, 2.5×10⁵ cells can be seeded one day before they are required for further use into 6 well plates so that they become 70-80% confluent on the day of transfection. Cells can then be transfected with LIPOFECTAMINE™/DNA complexes (Life Technologies). For transfection of BHK cells (gII⁺ or gH⁺), 1 μg plasmid DNA (in 100 μl of OPTI-MEM®, Life Technologies) can be mixed with 10 μl of LIPOFECTAMINE™ lipid (in 100 ∥l of OPTI-MEM®) and the mixture left at room temperature for 30 mins. At the end of this time, the total volume can be made up to 1 ml with OPTI-MEM®, and the mixture applied to OPTI-MEM®-washed cells in a 6-well plate. Five hours later, the transfection mixture can be removed and 2 ml of growth medium containing 10% FCS added. The cells can be infected next day with 3 plaque-forming unit (pfu) per cell of DISC-HSV. At completion of the cytopathic process (24-36 hrs), virus can be harvested by scraping cells off the plate. For 293 cells, transfection can be performed in the same way except that cells can be infected with adenovirus e.g. wild type Adenovirus type 5, at a multiplicity of infection (MOI) of 2 per cell and viruses acn usually be harvested at 48-72 hrs. In both cases, cells can be collected by low-speed centrifugation in a bench-top centrifuge and resuspended in 1 ml of serum-free medium. Virus can be released from the harvested cells by sonication for 1 min in a waterbath sonicator. The cell lysates can be microfuged briefly to remove insoluble debris, and supernatants collected. Contaminating HSV and amplicon particles can be completely inactivated by incubating at 56° C. for 20 mins (which can be confirmed by plaque assay). LIPOFECTIN®/Integrin targeting peptide/DNA (LID) transfection complexes can be made as described by Hart et al, 1990). For transfection of BHK cells, complexes can be made by gentle mixing of three components: peptide 6 ([K16]GACRRETAWACG), plasmid DNA and LIPOFECTIN® (Life Technologies) in the weight ratio 0.75;4:1. For titrating rAAV, dilutions of crude cell lysate or purified virus were added to cells seeded on 6 well plates. Cells can be super-infected with either 3 pfu/cell or DISC-HSV or if 293 cells were used, adenovirus at a MOI of 2. GFP positive cells can be counted 16 hours after super-infection. If rAAV is to be titrated without super-infection of helper viruses, GFP positive cells can be counted two to three days after the initial rAAV infection.

Purification of rAAV for In Vivo Application:

For generation of highly purified stocks, 10⁷ BHK cells can be transfected (LIPOFECTAMINE™/DNA complexes) as described above using DISC-HSV as helper virus, and separate amplicons encoding the vector genome and the rep and cap genes (pHAV5 and pHAV7.3). 24-36 hours later, at completion of the lytic process, cells can be harvested by centrifugation, and lysed by repeated freeze-thaw. AAV vector stocks can be purified from sequential caesium chloride gradients, dialysed against HFPFS buffered saline, concentrated by ultrafiltration (Microcon 30), and heated to 56° C. for twenty minutes to inactivate residual DISC HSV. Transducing titre can be determined by co-infection of HeLa cells with triplicate serial dilutions of rAAVgfp and wild type Ad5 at a MOI of 2. 24 hours later, gfp+ cells can be scored fluorescence microscopy. The rAAV titre in a total of 2 mls of crude cell lysate can be for example 1×10³ tu per ml. After purification and ultracentrifugation, the titre in a total volume of 200 μl can be for example 1×10⁹ tu per ml.

Generation of Herpes Amplicon Plasmids:

Useful yield of rAAV particles can be obtained when both the rAAV vector sequence and rep and cap genes are incorporated in HSV amplicon vectors. A series of HSV-derived amplicon plasmids can be constructed. A recombinant DISC-HSV vector encoding the rAAV vector sequence in the gH locus can also be constructed (DISC-AV2.1). To obtain AAV rep and cap genes pucAAV1 (entire type-2 AAV genome) can be partially digested with PpuMI and then completely digested with SnaBI. This can generate two AAV TR-minus DNA fragments: a 4303 bp fragment (nt 191 to 4493 of wtAAV genome) which is inclusive of an intact rep and cap expression cassette, and a 4143 bp fragment (nt 351-4493 of wtAAV genome) in which the p5 promoter and the N-terminal sequence of rep is deleted. Both DNA fragments can be cloned into the HSV amplicon plasmid pW7TK to create pHAV7.3 and pHAV7.3del, respectively, pHAV-7.3del an be used as a rep- control during production of rAAV. Sequences encoding a green fluorescent protein (gfp) expression cassette can be cloned into pucAAV1 previously digested with PpuMI and SnaBI, so that there is no sequence overlap between the rAAV vector and AAV helper constructs encoding rep and cap. The TR-flanked cassette can subsequently be cloned into pW7TK to create pHAV5 or into pHAV7.3 to create pHAV-4.1 (in which both AAV rep and cap genes and the AAV vector sequence are incorporated in a single amplicon plasmid). In addition, the TR-flanked GFP cassette can be inserted into the deleted gH locus of DISC-HSV-1 to create DISC-AV2.1. Parallel constructs based on conventional plasmids can also be constructed (pTR-EGFP, PHAV 5.6 and pHAV9.8).

Production of rAAV from HSV Amplicons and DISC-HSV Helper Virus:

To test the capacity of hybrid HSV-AAV to generate transducing rAAV particles, permissive producer cells can be transfected with plasmid combinations and infected with sufficient helper virus to ensure replication and lysis in every cell. Recombinant viruses can be harvested at completion of the cytopathic process (24 to 30 hrs for DISC-HSV helper virus in BHK cells or 48 to 72 hrs for Ad helper virus in 293 cells). The amount of rAAV recovered can be titrated as gfp+ transducing units (tu) in crude cell lysates as described above. Highest yields acn be obtained using DISC-HSV as helper virus, and HSV amplicon constructs encoding the vector genome and the helper AAV genome either separately (pHAV-5 and pHAV-7.3), or combined on a single amplicon (pHAV-4.1). For the combination of pHAV-5 and pHAV-7.3, more than 1000 tu of rAAV can be produced for each transfected cell. Without being bound by theory, it is believed that incorporation of the AAV vector genome and the helper AAV genome on separate amplicons can both contribute to improved rAAV recovery. Less preferred is to incorporate both rAAV vector sequences and the AAV helper genome on the same amplicon (pHAV-4.1) or to incorporate the vector genome as part of the DISC virus itself (DISC-AV2.1). To improve transfection efficiency of the RHK producer cells (which averaged between 10%-15% with LIPOFECTAMINE™/amplicon DNA complexes), a known efficient LID vector system can be used to deliver both plasmids. By this method, 25% of cells can be transfected, and the yield of rAAV can be approximately 4000 tu per transfected cell, suggesting that the efficiency of transfection of each cell can also be enhanced.

In certain examples carried out so far, it was found that the yield of rAAV recoverable from cells transfected with separate HSV amplicon plasmids and infected with DISC as the helper virus can be much higher than that generated from the same constructs in 203 cells using Ad as helper, and can also be higher than that recovered from RHK cells transfected with conventional plasmids.

Preparation of rAAV for Transduction of Cells In Vivo:

For in vivo use, rAAV encoding gfp can for example be generated using pHAV-5 pHAV-7.3 and DISC virus as helper. After caesium gradient purification and ultracentrifugation, rAAV can be obtained at a titre in a total volume of 200 μl of about 1×10⁹ tu per ml measured by co-infection of HeLa cells with Ad). Transducing titres in the absence of Ad may be approximately 80 fold lower.

Vectors according to the present invention can also be applied for example in ways analogous to those described in specification WO 96/29421 (Lynxvale Ltd & Cantab Pharmaceuticals Research Ltd: Efstathlou, Inglis, and Zhang: ‘Vectors for gene delivery’) which is incorporated herein by reference in its entirely for all purposes, including methods of amplicon culture described therein.

The invention described herein is susceptible to further modifications and variations as will be apparent to the reader of ordinary skill in the field. The present disclosure is intended in extend also to combinations and subcombinations of the features mentioned or described in the foregoing escription including the appended claims, and in the cited publications. Documents cited herein are hereby incorporated by reference in their entirety for all purposes.

1. A preparation of infectious viral particles including particles which can act as helper virus for adeno-associated virus (AAV), and including particles comprising DNA (i) that includes at least one chosen nucleic acid sequence for delivery to target host cells, and further encoding proteins and replicating functions which together are sufficient, when said particles of said preparation infect first target host cells, for assembly and release, from said first target cells, of infectious recombinant AAV particles that comprise said chosen nucleic acid sequence, whereby said infectious recombinant AAV particles are able in turn to infect second target host cells, and cause expression of said DNA (I) in said infected second target host cells.

2. A preparation according to claim 1, comprising infectious herpesviral amplicons and/or infectious mutant herpesvirus having a mutant genome disabled in respect of a gene essential for production of infectious new herpesvirus particles, said preparation encoding proteins and replicating functions sufficient (in first target host cells when infected by said preparation) to allow assembly and release, from said first target host cells, of infectious particles of recombinant adeno-associated virus encoding said chosen nucleic acid sequence, for delivery to second target cells when infected by said recombinant adeno-associated virus so released from said first target host cells.

3. A preparation according to claim 1, wherein said viral particles are free of virus particles capable of producing infectious new virus particles (other than said recombinant AAV particles) in a normal host cell.

4. A preparation of recombinant infectious herpesviral particles and/or herpesviral amplicon particles which
(a) lack a gene function essential for production of infectious new herpesviral particles in a normal host cell, and
(b) comprise
(i) DNA heterologous to AAV, e.g. up to about 4.5 kb in size, flanked by ITR sequences of AAV, and
(ii) DNA encoding AAV rep and cap genes coded at least in part in a position other than flanked by AAV ITR sequences,
wherein both DNA (i) and DNA (ii) are positioned in relation to a herpesviral origin of replication (oriS) and optionally also a herpesviral packaging signal (pac) so that they are replicatable within a cell infected by the virus particles.
such that when said particles infect first target cells, being normal host cells, no infectious new particles of herpesvirus or of herpesviral amplicon are produced, but said first target cells infected with said virus particles can give rise to recombinant AAV particles comprising said DNA (i), packaged in AAV coat protein, and able, after their release from said first cells, to infect second target cells and cause expression of said DNA (i) in said infected second target cells, but not able to give rise to infectious new virus particles from said infected second target cells.

5. A preparation according to claim 4, in which said DNA (i) and DNA (ii) are encoded in the same herpesvirus particle or in the same herpesviral amplicon.

6. A preparation according to claim 4, in which said DNA (i) and the DNA (ii) are encoded by different herpesviruses or herpesviral amplicon particles

7. A preparation according to claim 6, in which said DNA (i) is encoded by a first herpesviral amplicon and said DNA (ii) is encoded by a second herpesviral amplicon.

8. A preparation according to claim 6, in which said DNA (i) and DNA (ii) are both encoded by a respective infectious mutant herpesvirus that has a mutant genome lacking a gene essential for production of infectious new herpesvirus particles.

9. A preparation according to claim 5, comprising infectious mutant herpesvirus lacking a gene essential for production of infectious new herpesvirus particles encoding said DNA (ii) and further comprising herpesviral amplicon particles encoding said DNA (i).

10. A preparation according to claim 6, comprising infectious mutant herpesvirus lacking a gene essential for production of infectious new herpesvirus particles encoding said DNA (i), and further comprising herpesviral amplicon particles encoding said DNA (ii).

11. A preparation according to claim 4, in which said DNA (i) and/or (ii) is/are encoded by the mutant herpesvirus, and inserted at a site of deletion of said essential gene.

12. A preparation according to claim 1, wherein said chosen nucleic acid sequence comprises a reporter gene, e.g. a gfp gene or LacZ gene, or a gene encoding a functional fragment thereof.

13. A preparation according to claim 1, wherein said chosen nucleic acid sequence further comprise additional heterologous nucleic acid, e.g. a tissue specific promoter, e.g. an albumin promoter or neuronal enolase promoter.

14. A preparation according to claim 1, in which said chosen DNA for delivery to said target cell encodes an antigen capable of evoking an immune response in a human or non-human animal.

15. A preparation according to claim 1, wherein said chosen nucleic acid sequence comprises a gene encoding a cytokine or other immunomodulatory protein, e.g. IL-2.

16. A preparation according to claim 1, wherein said chosen nucleic acid sequence comprises a gene encoding a therapeutic protein, e.g. factor IX.

17. A preparation according to claim 1, incorporating DNA which is heterologous to AAV, e.g. up to about 4.5 kb in size flanked by ITR sequences of AAV, and accompanied by DNA encoding the rep gene function of AAV to enable integration of said DNA into the DNA of said second target cells when infected by said recombinant AAV particles produced by said first target cells.

18. A preparation of infectious recombinant AAV (adeno-associated virus) genomes comprising heterologous DNA and packaged in AAV coat protein, producible by infection of a host cell with a preparation according to claim 1, and free of helper virus, e.g. free of replication-competent helper-virus, e.g. containing infectious mutant herpesvirus lacking a gene essential for production of infectious new herpesvirus particles.

19. A method of producing recombinant AAV genomes, e.g. free of replication-competent helper virus, comprising heterologous DNA and packaged in AAV coat protein. comprising the steps of:
(i) providing a herpes virus comprising a genome lacking a gene essential for production of infectious new herpesvirus particles, and grown by culture on cells made recombinant and able to express the function of the viral gene that is lacking in the herpesviral genome:
(ii) providing a herpesviral amplicon comprising a rep and cap gene of AAV, and further comprising heterologous DNA desired to be incorporated in a recombinant AAV particle, and ITRs positioned so as to flank said heterologous DNA;
(iii) using the herpesvirus from (i) and the amplicon from (ii) to infect cells that do not express the function of said essential gene lacking in the herpesviral genome, and
(iv) harvesting from the cells infected in (iii) said recombinant AAV genomes comprising said heterologous DNA and packaged in AAV coat protein, preferably free of replication-competent helper virus.

20. A preparation of recombinant AAV particles, comprising a preparation of recombinant AAV genomes comprising heterologous DNA, e.g up to about 4.5 kb in size, flanked by ITR sequences of AAV and packaged in AAV coat protein, free of helper virus, and/or free of adenovirus, and/or free of infectious replication-incompetent helper-virus, and/or containing replication-defective herpesvirus but free of replication-competent herpes virus or other helper virus.

21. A method of monitoring gene expression in a subject or in a culture of cells comprising the steps of:
(i) administering to said subject or to said culture a preparation according to claim 1, wherein said DNA for delivery to target cells comprises a reporter gene; and thereafter
(ii) monitoring cells from said subject or said culture for expression of said reporter gene in vivo or in vitro, by detection of a corresponding reporter gene product, e.g. by ELISA or detection of fluorescence, e.g. by fluorescence microscopy.
